(19) United States
(12) Patent Application Publication
(10) Pub. No.: US 2023/0341400 A1
(43) Pub. Date: Oct. 26, 2023

(54) Method to identify, isolate and quantify a biomarker for the degradation of the lysosomal alpha-glucosidase, GAA, and to detect and isolate related glycosylated proteinsin vitro and in vivo

(71) Applicant: Allen Ketcik Murray, Irvine, CA (US)

(72) Inventor: Allen Ketcik Murray, Irvine, CA (US)

(73) Assignee: Allen Ketcik Murray, Irvine, CA (US)

(21) Appl. No.: 18/071,907

(22) Filed: Nov. 30, 2022

Related U.S. Application Data
(60) Provisional application No. 63/283,513, filed on Nov. 28, 2021.

(51) Int. Cl. G01N 33/573; G01N 33/68

(52) U.S. Cl. CPC G01N 33/573; G01N 33/6893; G01N 2333/934

(57) ABSTRACT

In studies on the degradation of glycogen by rhGAA, a glycosylated protein core material was found which consists of about 5-6 percent of the total starting glycogen. There was an additional 25 percent of the glycogen unaccounted for based on glucose released. After incubation of glycogen with rhGAA several oligosaccharides are only detected on HPAEC-PAD if the medium is first boiled in 0.1N HCl or incubated with trypsin. The characteristics of the in vivo serum material are identical to the material in the in vitro incubation medium. One oligosaccharide cannot be further degraded by rhGAA, Several masked oligosaccharides in serum contain m-inositol, e-inositol, and sorbitol as the major carbohydrates. The biomarker is not present in the serum of Pompe mice not on ERT, but it is present in those on ERT, so it is a biomarker of GAA degradation of lysosomal glycogen. The biomarker has also been detected in plasma.

This application includes it their entirety two publications by Allen Ketcik Murray:
1. The Release of a Soluable Glycosylated Protein from Glycogen by Recombinant Lysosomal αGlucosidase (rhGAA) In Vitro and Its Presence in Serum In Vivo Allen K. Murray 1,2. Biomolecules 2020, 10, 1613; doi:10.3390/biom10121613
Murray, Allen K., 2021, The Action of Recombinant Human Lysosomal αGlucosidase (rhGAA) on Human Liver Glycogen: Pathway to Complete Degradation, Int. J. Transl. Med. 2021, 1, 381-402. doi.org/10.3390/ijtm1030023.

### INTRODUCTION

Glycogen, the storage form of glucose in animals, is a complex polymer of glucose consisting of chains of α-1,4 linked glucose residues with α-1,6 linked branch points about every 12 residues, and it consists of 12 layers with a molecular weight up to 107 kDa [1]. Glycogen is organized into spherical particles of size similar to the calculated size of the spherical model [2,3]. At the reducing end is a protein, glycogenin, which functions as a self-glycosylating primer for the synthesis of the molecule. Glycogen is primarily degraded by phosphorylase for the linear chains and a debranching enzyme to cleave the 1,6 branches. Glycogen and these degradative enzymes are cytoplasmic in all cells but are most abundant in liver and muscle. In addition to the cytoplasmic components of glycogen metabolism, there is a lysosomal α-glucosidase which degrades glycogen in lysosomes. About 1-2% of the cell's glycogen is localized in the lysosomes.

In 1963, H. G. Hers reported the deficiency of the lysosomal α-glucosidase (GAA) in Type II glycogenosis which became known as Pompe disease, as well as acid maltase deficiency [4]. His initial report demonstrated the inability to degrade glycogen, but did not specifically report the deficiency of α-1,6-glucosidase activity. In 1964, the lysosomal α-glucosidase was shown to have α-1,6-glucosidase activity in dog liver [5]. In 1970, Brown et al. reported the absence in α-1,6-glucosidase in human Pompe disease tissues [6]. The enzyme was shown to be capable of transglucanase, transglucosylation, maltase and glucamylase activities in addition to α-1,4-glucosidase and α-1,6-glucosidase activities [7-11].

The complexity of the structure of glycogen and the different activities of the enzyme are often not included in discussions about the deficiency in Pompe disease. As mentioned, GAA has α-1,4-glucosidase, α-1,6-glucosidase, endoglucanase and glucosyltransferase activities which facilitate its degradation of glycogen [7-11]. This means it can remove a terminal α-1,4-linked glucose, a terminal α-1,6 linked glucose, it can transfer a glucose to another molecule or it can cleave internally in a glucose chain and potentially attach it to another molecule.

The activities of phosphorylase and glycogen debranching enzyme are well known as are the multiple activities of the lysosomal α-glucosidase. However following enzyme replacement therapy (ERT) in the genetically deficient Pompe mousee and in patient biopsy tissue there appears to be residual carbohydrate material which is also present in the cytoplasm. So the original question was to determine if the recombinant lysosomal α-glucosidase (rhGAA) can completely degrade glycogen. This involved incubating isolated human glycogen and commercially obtained bovine glycogen with rhGAA for long time periods until no more glucose was released. The result was a water insoluble residue at the bottom of the incubation tubes. A residue of glycosylated protein, which is glycosylated primarily with inositol and sorbitol, iditol and has minor constituents of glucose, galactose, and mannose, as well as galactosamine and glucosamine, was identified. The mass of which consists of about 5-6% of the initial glycogen in the incubation tube [12].

This present work is the result of an unexpected observation of that earlier work. The mass of glucose released by rhGAA and the residual glycosylated protein do not equal the mass of starting glycogen so about another 25% of the glycogen was unaccounted for. It is this unaccounted for glucan and a glycosylated protein containing primarily inositol and sorbitol which are the subjects of this patent. About 70-75% of the mass of glycogen is released as glucose by the action of rhGAA in vitro. After approximately four days of in vitro incubation of glycogen with rhGAA, the glucose released reaches a plateau and no more glucose is released. No carbohydrate was detected in the medium that eluted after glucose by HPAEC-PAD on a CarboPac PA1 column. If the medium was first boiled in 0.1 N HCl for 30 min, a number of oligosaccharides were detected. Incubation with trypsin also exposed oligosaccharides for detection. It appears that this is a case of a protein masking carbohydrate which is unusual but some cases have been reported [13,14]. The soluble glycosylated protein in the medium is bound by Dowex 50W, which is evidence of binding as a charged entity such as a protein but it is not bound by concanavalin A which binds carbohydrates containing glucose or mannose, including glycogen [15]. Based on these characteristics and the possibility of the involvement of lysosomal exocytosis, serum was investigated and this soluble glycosylated protein was found to be present in serum.

Incubation Medium Analysis

Demonstration of the oligomers in glycogen and their relationship to degradation by rhGAA

is shown in FIG. 1A-C. FIG. 1A shows the incubation medium from rhGAA degradation of Control 2 glycogen at 4 and 6 days of incubation, which only shows glucose released in the lower two plots, after four days no more glucose was released for up to 14 days. However, when the medium was extracted with 0.1 N HCl at 100° C. for 30 min, oligosaccharides were detected as shown in the middle two plots. Then, when the medium was treated with 2 N TFA for 2 h at 100° C. to degrade oligosaccharides to monosaccharides, the result was the surprising appearance of more oligosaccharides particularly with Control 2 glycogen in the 6 day medium as shown in the top two plots. It should be noted that at day 4, additional medium and enzyme were added. This particular glycogen sample, Control 2, appears to have a higher degree of complexity as shown in Murray [9]. This result was not as apparent in other glycogen samples. This particular glycogen sample was obtained from an organ donor who was maintained on life support until the organs could be harvested so this liver tissue was then frozen immediately. Some suggest that degradation of glycogen is apparent at 15 min after death. These results, and others, indicates that changes begin to take place very soon after death which may be why this glycogen may be somewhat different than other glycogens from tissue obtained at autopsy. FIGS. 1B and C demonstrate that the oligomers in TFA extracts can be degraded by rhGAA, however this is most apparent in the day 4 samples, which is due to the fact that the day 4 samples contains incubation medium from the beginning of the experiment. There are shifts in the retention times of residual peaks which appear to correspond to the residual material originally obtained from rhGAA degradation. The identification of the fraction which is released by the 0.1 N HCl contains about 18% of the mass of the initial glycogen sample. The characteristic which results in the appearance of the oligosaccharides on HCl extraction of the medium is of interest since this was totally unexpected. The residual material fractions all contain protein.

The reaction mixture from rhGAA degradation of Control 2 glycogen was subjected to the scheme shown in FIG. 2A. At each of the six steps, the sample was analyzed and the results are shown in FIG. 2B.

Characteristics of the Glycogen Fraction that is not Degraded by rhGAA

1. HPAEC-PAD does not reveal any significant peaks that elute after monosaccharides. Which indicates no carbohydrates with ionizable hydroxyl groups are present?

2. Extraction with 0.1 N HCl at 100° C. for 30 min reveals maltooligosaccharides from DP 2 to about 18 on HPAEC-PAD.

3. The material in the incubation medium binds to a Dowex 50W ion exchange column and elutes in 2.0 N NH4OH. This is indicative of binding by a charged species such as protein or amino acids. After taken to dryness, it can be extracted with 0.1 N HCl at 100° C. to reveal the maltooligosaccharides.

4. Incubation with amyloglucosidase does not do anything to the samples.

5. Incubation with trypsin reveals some smaller oligosaccharides that elute in the region of up to about DP 4 and one at about DP 7 or 8. Additionally, trypsin treatment before HCl extraction appears to facilitate the appearance of more larger oligosaccharides. This is indicative of oligosaccharides being released or their appearance facilitated by the removal of protein. Incubation with concanavalin A does not appear to bind the material. This indicates the absence of exposed glucose or mannose residues, including glycogen, which would be bound by the concanavalin A protein [15].

Summary of Characteristics
Lack of chemical detection of ionizable hydroxyls of carbohydrate.
Lack of biological recognition of carbohydrate by rhGAA, concanavalin A, or amyloglucosidase.
Binding to Dowex 50W indicative of a charged species.
Exposure of carbohydrate by incubation with a protease (trypsin).

These characteristics led to the conclusion that the material contains carbohydrate material which is masked by protein. There are reports in the literature of carbohydrate masked by protein. Since the material was not detected to be carbohydrate chemically, or by glycosidases and concanavalin A, it is possible that it is not recognized by the biological system. It was considered to be possible that it could be released outside the cell by the lysosomal exocytosis mechanism in which the lysosomal membrane fuses with the cell membrane and the lysosomal contents are expelled from the cell [16,17]. This has been shown for the export of stored glycogen from Pompe mouse cells in culture [18,19] and for the release of lysosomal enzymes in urine [20]. If that were the case, then it seemed reasonable that this material might be found in blood or urine. Normal human serum was investigated and the material was found to be present, indicating that this may be part of the normal mechanism of degradation for lysosomal glycogen.

Serum Investigation

About 200 μL of blood was obtained from a fingertip needle stick of a normal individual and added to 300 μL of 0.9% NaCl in a conical 1.5 mL tube and immediately centrifuged for 10 min at 10,000×g and allowed to clot. The serum was then diluted 1:10 and 1:20 and analyzed by HPAEC-PAD directly as well as after extraction with 0.1 N HCl for 30 min at 100° C. The serum, HCl extract, and HCl extract following in vitro incubation of glycogen with rhGAA are shown in FIG. 3A. The peak labeled “Unknown” is present in all of the serum samples analyzed. However, there was significant variability in the oligosaccharide content of the HCl extracts between different serum samples from the same source from day to day. The presence of the oligosaccharides varied but the unknown peak was consistently the same. The unknown peak did vary in magnitude with respect to the time of day the sample was obtained and with respect to physical activity of the subject. The clot at the bottom of the serum but above the red blood cells was extracted. The HCl extract of the clot is characterized by abundant oligosaccharides. Since the clot contains fibrin and as a protein its function is to bind proteins or other components of blood, it is not surprising that the clot bound oligosaccharides, which appear to be conjugated to protein. Following HCl extraction, the oligosaccharides are readily degraded in vitro by rhGAA but the rhGAA does not degrade the unknown. The degradation of the oligosaccharides results in an increase in the size of the unknown peak.

The HCl extract of the clot shown in FIG. 3A contains almost the full array of maltooligosaccharides from DP2-16. The small peaks between the oligosaccharide peaks in FIG. 3A are the glycosylated protein peaks associated with the oligosaccharides [20], as well as the unknown.

The rhGAA degradation of the oligosaccharides in the clot extract exposes the unknown and leaves the small peaks as well as some of the oligosaccharide peaks, which is shown in FIG. 3B. The oligosaccharide peaks would likely not be there if a longer incubation was used. The chromatograms for the clot HCl extract before and after rhGAA degradation are shown in FIG. 4A. The retention times for components can vary in different solutions as is the case with the HCl extract of incubation medium and the HCl extract of serum or the clot. This can be due to salt concentrations and other components of the solution. Therefore, to establish the identity of the material from the two different sources, equal volumes of both solutions containing the same amount of the unknown were combined and the mixture was chromatographed. In this case, the result was only one peak, which was symmetrical with no leading or trailing shoulders indicative of identity of the material from both sources as shown in FIG. 4B. In this case, the Control 2 glycogen HCl extract was from day 8 of a rhGAA degradation of glycogen where the unknown was the only oligosaccharide remaining. It should be pointed out that there may be slight differences in absolute retention times of components but not in relative retention times. This is due to the fact that some chromatograms were obtained with only the electrochemical detector in use and other times with the addition of the photodiode array detector, which is in line ahead of the electrochemical detector, resulting in a slight delay in elution of peaks detected by the electrochemical detector.

The rhGAA degradation of the oligosaccharides in the clot extract exposes the unknown and leaves the small peaks as well as some of the oligosaccharide peaks, which is shown in FIG. 3B. The oligosaccharide peaks would likely not be there if a longer incubation was used. The chromatograms for the clot HCl extract before and after rhGAA degradation are shown in FIG. 4A. The retention times for components can vary in different solutions as is the case with the HCl extract of incubation medium and the HCl extract of serum or the clot. This can be due to salt concentrations and other components of the solution. Therefore, to establish the identity of the material from the two different sources, equal volumes of both solutions containing the same amount of the unknown were combined and the mixture was chromatographed. In this case, the result was only one peak, which was symmetrical with no leading or trailing shoulders indicative of identity of the material from both sources as shown in FIG. 4B. In this case, the Control 2 glycogen HCl extract was from day 8 of a rhGAA degradation of glycogen where the unknown was the only oligosaccharide remaining. It should be pointed out that there may be slight differences in absolute retention times of components but not in relative retention times. This is due to the fact that some chromatograms were obtained with only the electrochemical detector in use and other times with the addition of the photodiode array detector, which is in line ahead of the electrochemical detector, resulting in a slight delay in elution of peaks detected by the electrochemical detector.

Masking of Carbohydrate by Protein

From the initial observation of the in vitro degradation of glycogen, that the apparent absence of oligosaccharides in the incubation medium could be overcome by boiling in 0.1 N HCl for 30 min or by trypsin, the concern became one of the comparison of the methods. An overnight or 24 h incubation with trypsin did not reveal as much of the terminal oligosaccharide, which is not degraded by GAA as was released by the HCl treatment. However, longer trypsin incubation releases much more of the material as shown in FIG. 5A. It is also apparent that the HCl treatment results in a shift to a slightly longer retention time as well as variable release of the other oligosaccharides. The chymotrypsin treatment appears to release more oligosaccharide material, although it takes longer, as shown in FIG. 5B. The products released are reproducible. The first two peaks with retention times of about 9 and 11 min as well as the last oligosaccharide with a retention time of 26 min are the major ones and as a result the ones of major interest. They all still have peptide material attached as shown in plots which show both the electrochemical detector and the absorbance at 280 nm. The retention times do shift slightly when other proteases are used due to the different specificity of which amino acids the proteases cleave.

From the initial observation of the in vitro degradation of glycogen, that the apparent absence of oligosaccharides in the incubation medium could be overcome by boiling in 0.1 N HCl for 30 min or by trypsin, the concern became one of the comparison of the methods. An overnight or 24 h incubation with trypsin did not reveal as much of the terminal oligosaccharide, which is not degraded by GAA as was released by the HCl treatment. However, longer trypsin incubation releases much more of the material as shown in FIG. 5A. It is also apparent that the HCl treatment results in a shift to a slightly longer retention time as well as variable release of the other oligosaccharides. The chymotrypsin treatment appears to release more oligosaccharide material, although it takes longer, as shown in FIG. 5B. The products released are reproducible. The first two peaks with retention times of about 9 and 11 min as well as the last oligosaccharide with a retention time of 26 min are the major ones and as a result the ones of major interest. They all still have peptide material attached as shown in plots which show both the electrochemical detector and the absorbance at 280 nm. The retention times do shift slightly when other proteases are used due to the different specificity of which amino acids the proteases cleave.

Fraction Collection and Evidence of Protein Masking by Carbohydrate in Serum

The effect of doubling the concentration of trypsin used as well as chymotrypsin was tried.

In 48 h incubation, doubling the trypsin concentration did not have a noticeable effect on the result. Chymotrypsin was more effective at the same concentration as trypsin. Proteinase K was also tried but there is a problem with proteinase K since it contains a number of oligosaccharides in the enzyme preparation which makes it problematic for collection of fractions. Six fractions were collected from a trypsin incubation mixture which are labeled 1-6 in the top panel of FIG. 6. As mentioned earlier fractions 1, 2, and 6 are quantitatively the major ones of interest. These fractions were collected from multiple chromatographic runs into the same tubes. Below the top chromatograms, the parallel treatments of fractions 1 and 2 are shown. The fractions were partially concentrated on a Speed-Vac and then dialyzed against water at room temperature overnight using a 500 MWCO membrane in 1 mL dialyzers. Following dialysis, no oligosaccharides were detected as shown in the top chromatogram for each sample. There were a few small peaks at about 2-3 min retention time which represent sugar alcohols and a small peak at about 3.5 min which represents glucose galactose and mannose are not separated from glucose under these conditions. Next, the fractions were hydrolyzed in 2 N trifluoroacetic acid at 100° C. for two hours and then taken to dryness in the Speed-Vac. The fractions were then made up to 1.0 mL in water. Chromatography again on the PA1 column revealed increased monosaccharides but no oligosaccharides as shown in the second chromatogram for each fraction. The fractions were then analyzed by chromatography on an MA1 column which revealed inositol, sorbitol, hexoses, and xylose in 2-2 (Shown in FIGS. 7 and 8). The samples were then passed over a Dowex 50W column to remove amino acids. Following hydrolysis in TFA, the material which passed through was then analyzed by chromatography on the MA1 column which indicated increased inositol and sorbitol (shown in FIGS. 7 and 8). This was likely due to additional hydrolysis on the resin which is not uncommon. Since it was apparent that everything had not been hydrolyzed the samples were then hydrolyzed in 4 N TFA at 120° C. for two hours, dried on a Speed-Vac and made up to 1.0 mL in water. Subsequent chromatography on an MA1 column revealed significantly more m-inositol, e-inositol, sorbitol, and xylitol (tentative) as well as hexoses and less xylose in 2-2 as shown in the bottom chromatogram for each fraction. During acid hydrolysis to release carbohydrates, although some carbohydrates are released with increasing time, others may be degraded. In this case, during the 4 N TFA hydrolysis, some glucose and a significant amount of xylose are degraded with increased time. This final TFA hydrolyzate is shown for both fractions in the bottom panel of FIG. 6.

fractions apparent when the dialyzed sample is chromatographed. However, TFA hydrolysis of the fractions demonstrates that the material was present but that it was masked. Therefore, it appears that the initial in vivo material from serum or the incubation medium from in vitro rhGAA degradation is masked by protein. After proteolysis with enzymes, there apparently is still enough peptide material attached to mask the carbohydrate after dialysis. It may be possible to remove more peptide material by using proteases with different specificities. It appears that after initial hydrolysis with 0.1 N HCl at 100° C. for 30 min to expose the carbohydrate, the removal of salt by dialysis then permits a configuration change to again mask the carbohydrate. This is the case for both the material from the in vitro rhGAA incubations and the in vivo material isolated from serum.

It is apparent from this sequence that the initial fractions from the collection in the 150 mM NaOH/NaOAc elution medium are altered by the dialysis to remove the salt. Those peaks are then not apparent when the dialyzed sample is chromatographed. However, TFA hydrolysis of the fractions demonstrates that the material was present but that it was masked. Therefore, it appears that the initial in vivo material from serum or the incubation medium from in vitro rhGAA degradation is masked by protein. After proteolysis with enzymes, there apparently is still enough peptide material attached to mask the carbohydrate after dialysis. It may be possible to remove more peptide material by using proteases with different specificities. It appears that after initial hydrolysis with 0.1 N HCl at 100° C. for 30 min to expose the carbohydrate, the removal of salt by dialysis then permits a configuration change to again mask the carbohydrate. This is the case for both the material from the in vitro rhGAA incubations and the in vivo material isolated from serum.

Monosaccharide Composition of Fractions

The three monosaccharide chromatograms for Fraction 1(2-1) after hydrolysis in 2 N TFA, followed by passage through a Dowex 50W column and hydrolysis in 4 N TFA are shown in FIG. 7. After each step, the dried sample was made up to 1.0 mL. Since 25 μL samples were injected on the column in each case, the losses between steps were minimal. The increasingly broad injection peak is indicative of samples containing protein since proteins and amino acids are not retarded on the column. The three monosaccharide chromatograms for Fraction 2(2-2) after hydrolysis in 2 N TFA followed by passage through a Dowex 50W column and hydrolysis in 4 N TFA are shown in FIG. 8. The monosaccharide composition of the six fractions is shown in FIG. 9.

From the composition of the fractions it appears that the major components are two inositols, sorbitol, xylitol, and mannitol are relatively similar and that the variability occurs in the monosaccharides. However, it is important to keep in mind that these are the monosaccharide compositions of oligosaccharides that still have some peptide attached. On incubation with rhGAA, all of them except 2-2 are degraded with an increase in 2-2 and free glucose, which indicates that although glucose is only a minor constituent, it likely is in a critical position. This is suggestive that at least a portion of the other glycopeptides are being converted to 2-2. There is still peptide attached but it is not known if the peptide is the same for all of them so it is not yet possible to determine with absolute specificity the quantitative interrelationships. It is very likely that there are multiple glycosylation sites, each having a different monosaccharide composition as will be discussed later.

The question of whether these in vivo fractions are intact components of glycogen or whether they have undergone some modification by GAA, or any other enzymes, is an open question since GAA does have glucanase, glucantransferase, and glucosyltransferase activities under the same conditions in which it has glucosyl hydrolase activity [4-9]. There is a commonly held belief that GAA only breaks glycogen down to glucose but it breaks down glycogen to some oligosaccharides which then are later degraded to glucose [9].

The carbohydrate composition of these soluble glycosylated proteins unique by consisting The carbohydrate composition of these soluble glycosylated proteins is unique by consisting

mainly of inositols and sorbitol with some iditol. Inositol and sorbitol are not known to be found on any other protein. Literature searches do not reveal any glycosylated proteins published with these as the major carbohydrate. In fact, a search does not reveal any publication of a glycosylated protein mainly of inositols sorbitol with some iditol. Inositol sorbitol are known to found on any other protein. Literature searches do not reveal any glycosylated proteins published with these as the major carbohydrate. In fact, a search does not reveal any publication of a glycosylated protein with sorbitol.

FIG. 10 shows the HPAEC-PAD chromatograms of the 0.1 N HCl extracts from the serum of

Pompe mice that did not receiver ERT and the serum of three Pompe disease patients that are on ERT. These results are what would be expected if the unknown peak of interest (2-2 in FIG. 6) is really a terminal degradation product of GAA degradation of glycogen. The unexpected result in FIG. 10 is the large peak at about 18 min retention time in the serum of the Pompe mice. The retention time is what would be expected for maltoheptaose. The peak was collected and incubated with trypsin, which resulted in several peaks with shorter retention times. Each of those peaks was collected, hydrolyzed, and monosaccharide composition determined. Each had a different composition, but combined, they consisted of the inositols, iditol, and sorbitol with lesser amounts of glucose, galactose, and mannose. So, they appear to be related to the other glycosylated proteins from glycogen. The fact that this peak is present in the serum of Pompe mice not on ERT and absent from the serum of Pompe patients on ERT also implies that it is related. The serum of Pompe mice on ERT will be investigated as soon as it is available. It is unclear how this peak may be related to glycogen but based on its presence in the serum of Pompe mice not on ERT, its absence from normal serum or serum of Pompe patients on ERT and the similarity of its carbohydrate composition to the other soluble glycosylated proteins, it appears that it is related.

A summary of the various fractions isolated following in vitro degradation of glycogen by as well as the fractions isolated from normal serum, in vivo, is shown in FIG. 11.

### ABBREVIATIONS

BSA bovine serum albumin
ERT GAA enzyme replacement therapy Lysosomal α-glucosidase
rhGAA recombinant human lysosomal α-glucosidase
HPAEC-PAD high performance anion exchange chromatography-pulsed amperometric detection
HPLC GLC TFA PAS
high performance liquid chromatography gas liquid chromatography
trifluoroacetic acid periodic acid Schiff stain.

1. Materials and Methods

Glycogen Substrates

Sigma, Type IX bovine liver glycogen, SigmaAldrich, St. Louis, MO, USA, is extracted by the method of Bell and Young, [21] which involves boiling and TCA precipitation of proteins at elevated temperature. This method is quite harsh compared to the method of isolation of the human glycogen in this report. All chemicals were of Reagent Grade or higher. Concanavalin A, monosaccharide and oligosaccharide standards and TFA were purchased from Sigma Aldrich, St. Louis, MO, USA. Dowex 50W was obtained from Bio-Rad, Hercules, CA, USA.

2.2. Human Glycogen Samples

Human glycogen samples were extracted by the method of Mordoh, Krisman, and Leloir [22] with the addition of five freeze-thaw steps to ensure the rupture of lysosomes. This method was chosen because it was reported that the isolated glycogen appeared to be identical to native glycogen isolated from liver as judged by its rate of sedimentation and its appearance under the electron microscope. Glycogen isolated by this method has been shown to be paracrystalline [23]. The glycogens were characterized for a number of parameters including average chain length, protein content, amino acid composition, RNA content, phosphate content, β-amolysis, iodine absorbance, interior chain length, and external chain length [24, 25]. The protein content was less than one percent for two of the three samples. All glycogens were hydrated for at least 18 h before incubations. Glycogen solutions were never frozen.

Source

Autopsy liver tissue from an 18-month-old female with Pompe disease (type II glycogenosis) and liver tissue from two adult male accident victims. The Pompe liver and the Control 1 liver were obtained at autopsy. In the case of Control 2, the patient was an organ donor on life support so the liver tissue was obtained immediately on termination of life support. All liver tissue was stored at −76° C. until the glycogen isolation. The case of the Pompe disease patient and an enzyme replacement trial with lysosomal α-glucosidase linked to low density lipoprotein has been previously reported [26]. The IRB approval was UC Irvine, UCI/2008-6631, and the genomic analysis of patients was

reported [27].

Enzyme Assays

Recombinant human GAA (rhGAA) was provided by Sanofi Genzyme, Framingham, MA, USA which is the 110 kDa precursor which is converted to the mature form in the tissue in ERT. Assay mixtures consisted of 1 mL volume containing 500 μg or more of glycogen as indicated, 50 mM sodium acetate buffer, pH 4.6, and 10 μL or 25 μL of rhGAA (5 μg/μL) as indicated. The reactions were incubated at 37° C. under toluene to prevent microbial growth. At various time points, as indicated in the figures, the reaction mixture was mixed on a vortex mixer, then centrifuged at 16,000×g for 5 min to precipitate any insoluble material. Then, a 100 μL or 200 μL aliquot was extracted and boiled for 5 min. The sample was then centrifuged at 16,000×g for 5 min to precipitate any insoluble material and the supernatant was analyzed for carbohydrates by HPAEC-PAD on a PA1 column. The remaining incubation mixture was mixed on a vortex mixer and returned to the water bath.

2.5. Carbohydrate Analysis

HPAEC-PAD was performed on a Dionex DX-600 ion chromatograph using a CarboPac PA1 column. (Thermo Fisher Scientific, Dionex, Thermo Elecdtron North America, LLC, Madison, WI, USA) The eluent was 150 mM sodium hydroxide, isocratic from 0 to 5 min, then a linear sodium acetate gradient from 5 to 25 min going from 0 to 57% 500 mM NaOAc in 150 mM NaOH at a flow rate of 1 mL/min. Fractions of 0.25 mL were collected using a Gilson 201 fraction collector. Fractions were partially reduced in volume on a Speed Vac to a volume less than 1.0 mL and then dialyzed overnight against 18.3 megohm water in 1.0 mL chambers against a 500 MWCO membrane. Fractions were taken to dryness in a Speed-Vac. The fractions were then hydrolyzed with 2 N TFA at 100° C. for two hours after which they were taken to dryness in a Speed-Vac. If it was determined that hydrolysis was incomplete, as evidenced by changes on passage through a Dowex column, samples were hydrolyzed again with 4 N TFA at 120° C. for 1 to 4 h. Monosaccharides and sugar alcohols were determined using a CarboPac MA1 column with isocratic elution with 480 mM NaOH at a flow rate of 0.4 mL/min. The waveform for carbohydrate analysis had a potential of +0.1 V from 0 to 0.40 s, −2.0 V from 0.41 to 0.42 s, +0.6 V from 0.43 to 0.44 s, and −0.1 V from 0.44 to 0.50 s with integration from 0.20 to 0.40 s. Data analysis was performed using Dionex Chromeleon 6.60 software.

Protein Determination

Protein determination was by a modification of the method of Lowry et al. [28]. A control

experiment of protein determination on BSA showed no significant difference between samples of before and after hydrolysis for comparison.

### Legends for Figures

FIG. 1. (A) Incubation medium of Control 2 with rhGAA after 4 and 6 days, HCl extract of the same and 2N TFA hydrolyzate of same. (B) Incubation medium of Control 2 glycogen with rhGAA after 4 days: 2N TFA hydrolyzate and same incubated with rhGAA. (C) Incubation medium of Control 2 glycogen with rhGAA after 6 days: 2N TFA hydrolyzate and same incubated with rhGAA.

FIG. 2. (A) Extraction procedures for incubation medium from rhGAA degradation of Control 2 glycogen after no more glucose is released. HPAEC-PAD numbers 1-6 refer to chromatograms 1-6 in 2B. (B) Results of samples analysis at each of sic steps in 2A.

FIG. 3. (A) Glycogen HCl extract showing maltooligosaccharides DP2-16 and clot HCl extract with array of maltooligosaccharides and the Unknown. (B) Serum, serum HCl extract, and serum HCl extract after rhGAA incubation.

FIG. 4. (A) Clot HCl extract before and after degradation with rhGAA demonstrating the Unknown is not degraded. (B) HCl extract of Control 2 glycogen, HCl extract of clot following rhGAA degradation, and a mixture of equal parts of both extracts demonstrating

FIG. 5. (A) Release of peptide bound oligosaccharides by trypsin and 0.1 N HCl. The HCl treatment was at the beginning. The trypsin treatment was for the indicated time at 37° C. under toluene. (B) Release of peptide bound oligosaccharides by chymotrypsin showing carbohydrate in blue and protein in blac

FIG. 6. The top chromatogram shows the fractions collected. The three chromatograms on the left show fraction 1 after dialysis, after hydrolysis with 2 N TFA and after hydrolysis with 4 N TFA from top to bottom. The three chromatograms on the right show the same three treatments of Fraction 2. Carbohydrates: 1, myo-inositol: 2, epi-inositol: 3, Xylitol: 4, Sorbitol: 5, Manitol: 6, Glucose: 7, Xylose: 8, Galactose.

FIG. 8. The monosaccharide chromatograms for Fraction 2 (2-2) are shown from bottom to top for 2 N TFA hydrolyzate, Dowex 50 column, and 4 N TFA hydrolyzate.

FIG. 9. Monosaccharide composition of fractions.

FIG. 10. The 0.1 N HCl extracts of serum, 1:10 from two Pompe mice, age 8.7 months and three Pompe patients. The peak at about 18 min in the Pompe mouse serum is a glycosylated protein containing m-inositol, sorbitol and glucose as major components as well as galactose and mannose.

FIG. 11. Summary of the metabolites discussed and reference to the figures showing them. The top portion shows metabolites from the in vitro incubations and the bottom portion shows in vivo metabolites isolated from serum.

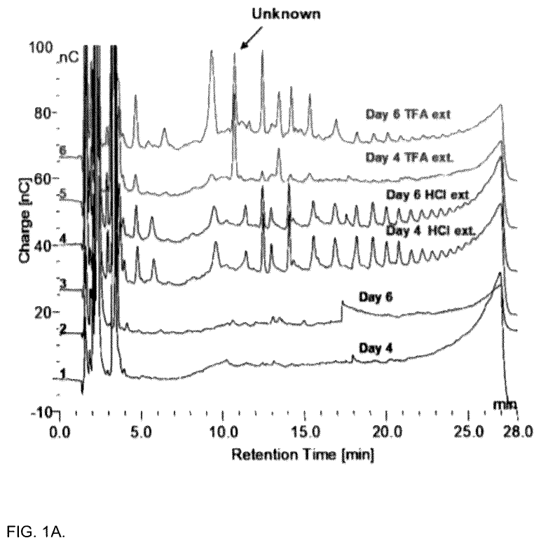

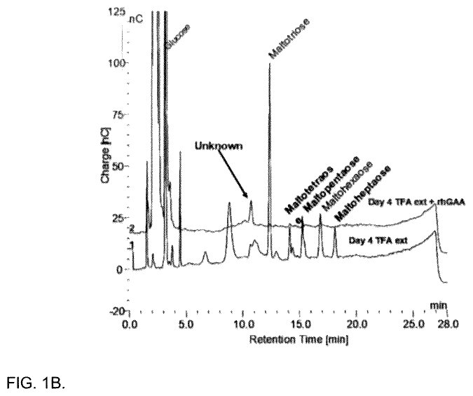

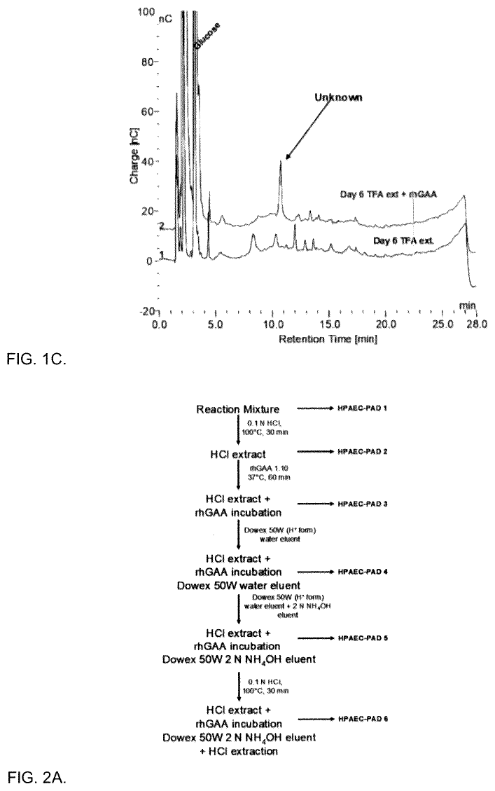

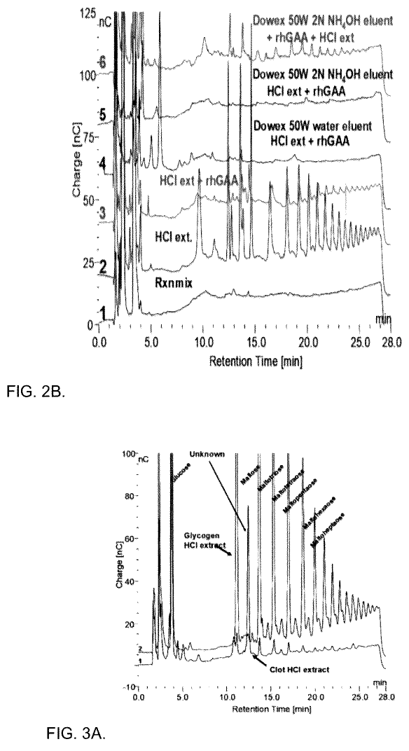

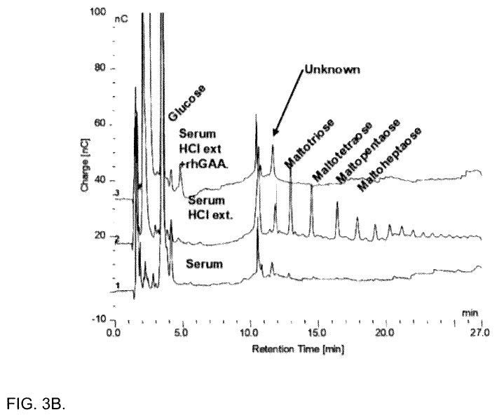

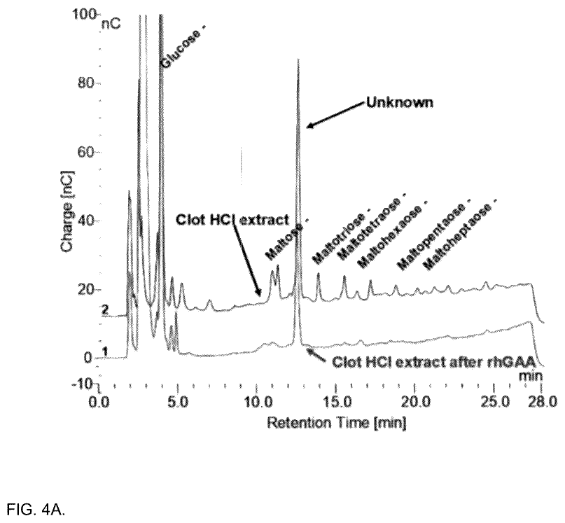

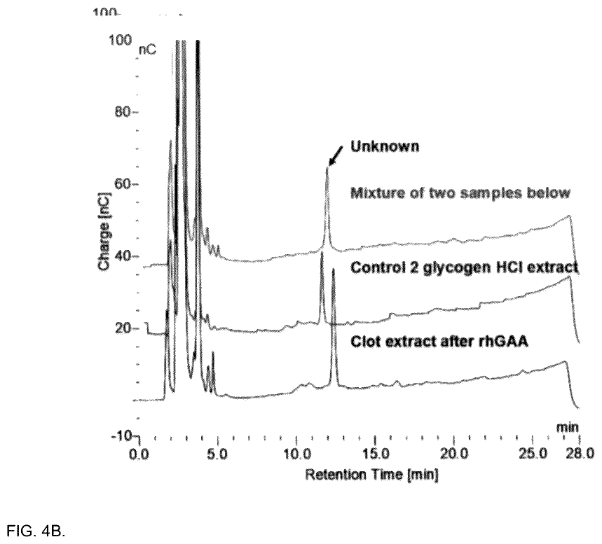

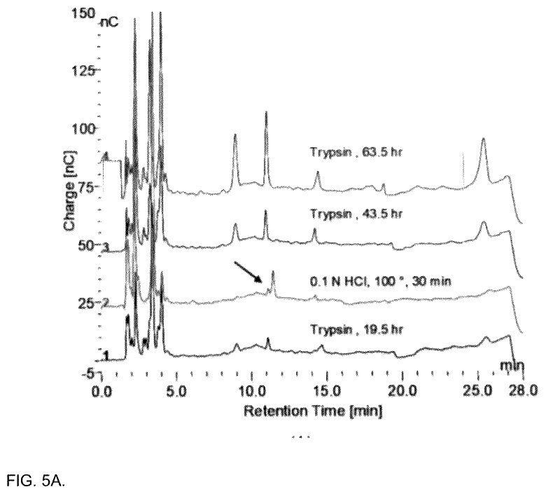

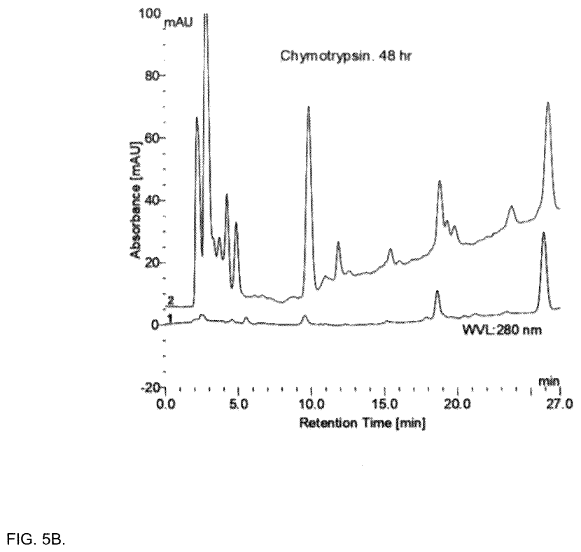

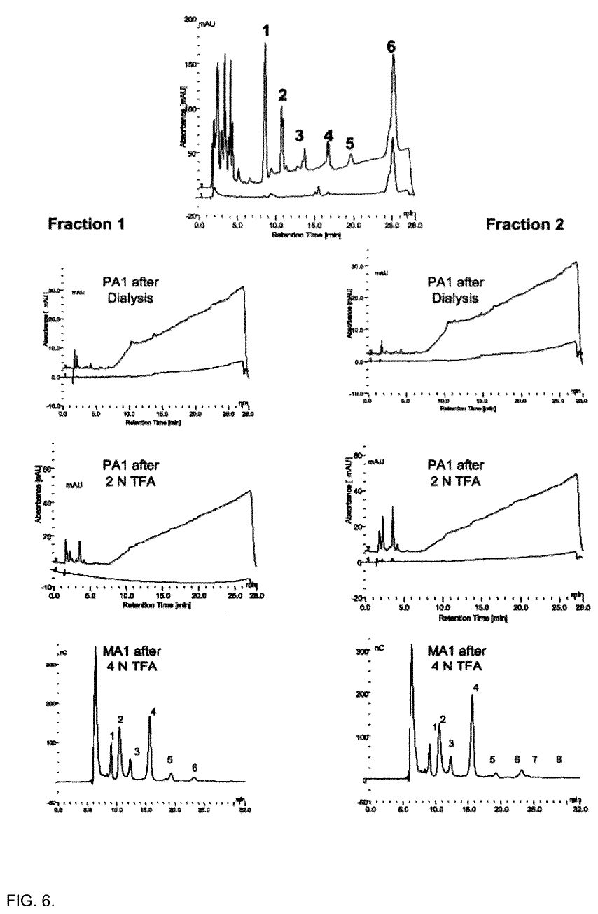

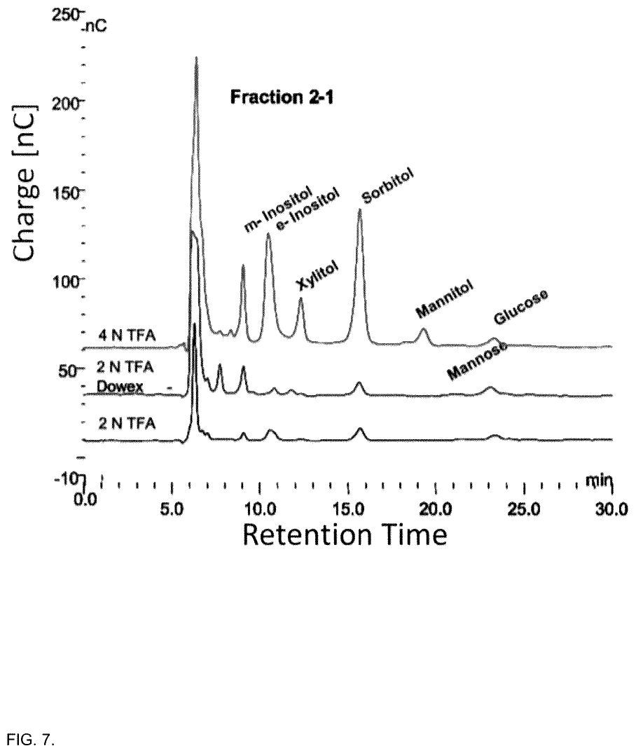

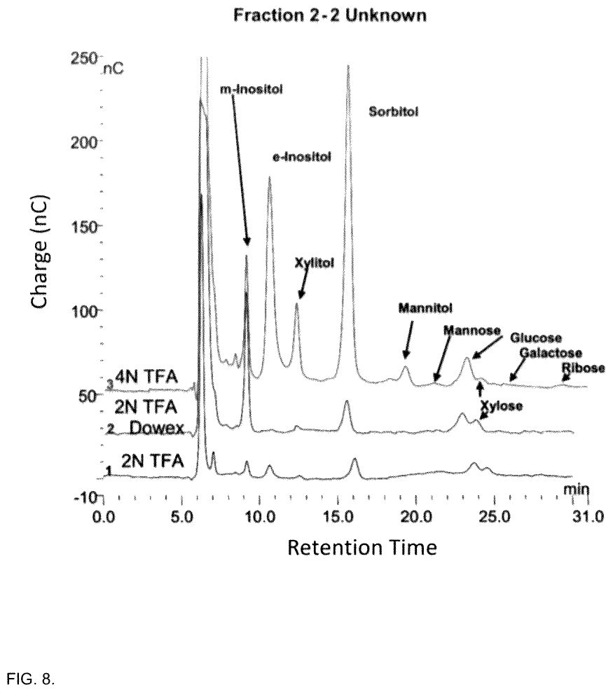

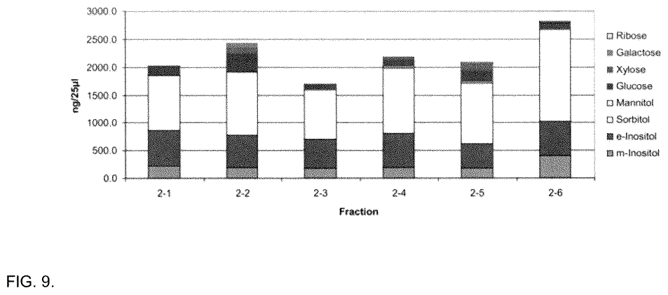

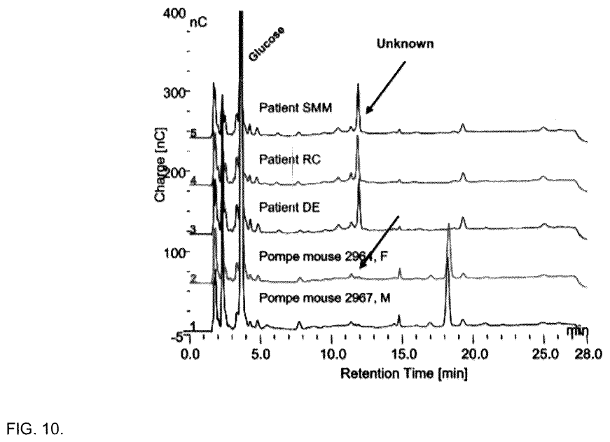

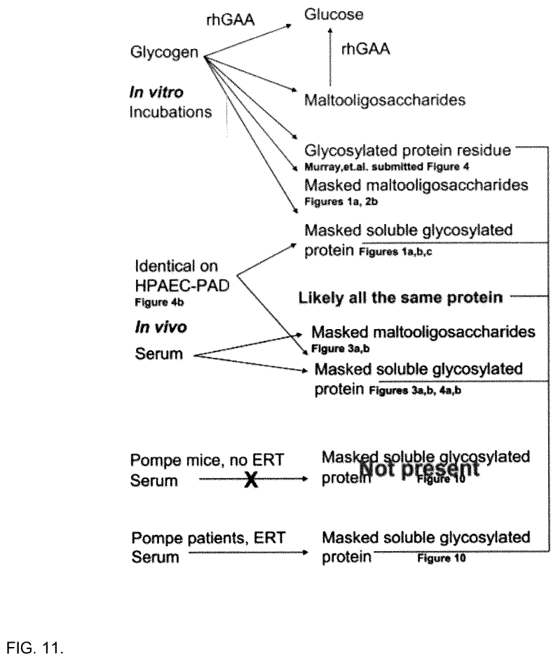

What is claimed is:

1. A method for detecting and isolating, by treatment with acid or proteases, a glycosylated protein which is a terminal degradation product from degradation of glycogen by the lysosomal àlpha-glucosidase, GAA or its recombinant form, rhGAA, in biological fluids, such as serum, plasma or others, for the purposes of any of the following:
A. Monitoring the dosage of rhGAA in patients on enzyme therapy
B. Monitoring the GAA activity of patients on gene therapy
C. Monitoring glycogen metabolism in humans or animals since all glycogen may ultimately be degraded by GAA and lysosomal exocytosis
D. Monitoring glycogen metabolism in human or animal athletes

2. A method for detecting and isolating, by treatment with acid or protease, glycosylated protein bound glucans, in biological fluids such as but not limited to serum or plasma, derived from glycogen and their degradation by glycosidases and amyloglucosidase in biological fluids for the purposes of any of the following:
A. Determination of the total carbohydrate in metabolic studies or monitoring of such carbohydrates for research or diagnostic purposes.
B. Monitoring glycogen metabolism in humans or animals since all glycogen may ultimately be degraded by GAA and lysosomal exocytosis
C. Monitoring glycogen metabolism in human or animal athletes

3. A general method by treatment with acid or protease, for detection of molecules consisting of carbohydrate components masked by protein for analytical, research, diagnostic or other purposes.
